(19) United States
(12) Patent Application Publication
(10) Pub. No.: US 2006/0188904 A1
(43) Pub. Date: Aug. 24, 2006

(54) Biosensors having single reactant components immobilized over single electrodes and methods of making and using thereof

(75) Inventors: Mihrimah Ozkan, San Diego, CA (US); Cengiz S. Ozkan, San Diego, CA (US); Mo Yang, Riverside, CA (US); Xuan Zhang, Riverside, CA (US); Shalini Prasad, Riverside, CA (US)

Correspondence Address: SMITH, GAMBRELL & RUSSELL, LLP (UC);SUZANNAH K. SUNDBY, 1850 M. STREET NW, # 800, WASHINGTON, DC, 20036, US

(73) Assignee: The Regents of the University of California

(21) Appl. No.: 11/331,161

(22) Filed: Jan. 13, 2006

Related U.S. Application Data
(63) Continuation of application No. 10/820,108, filed on Apr. 8, 2004.
(60) Provisional application No. 60/461,812, filed on Apr. 11, 2003.

(51) Int. Cl. C12Q 1/68; G01N 33/554; C12M 1/34; G01N 33/543; G01N 33/569

(52) U.S. Cl. 435/6; 436/518; 435/7.32; 435/287.2

(57) ABSTRACT

Disclosed herein are single reactant components immobilized over single electrodes and methods of making and using thereof. Devices, such as biosensors, comprising the single reactant components immobilized over single electrodes are also disclosed. Assays using the single reactant components immobilized over single electrodes are disclosed as well as databases comprising signature pattern vectors for reactant components.

### CROSS-REFERENCE TO RELATED APPLICATIONS

This application is a continuation of U.S. patent application Ser. No. 10/820,108 filed 8 Apr. 2004, pending, which claims the benefit of U.S. Provisional Patent Application No. 60/461,812 filed 11 Apr. 2003, which names Mihrimah Ozkan, Cengiz S. Ozkan, Mo Yang, Xuan Zhang, and Shalini Prasad as inventors, both of which are herein incorporated by reference in their entirety.

### STATEMENT REGARDING FEDERALLY SPONSORED RESEARCH OR DEVELOPMENT

This invention was made with Government support under Grant No. DMEA 90-02-2-0216, awarded by the Department of Defense (DOD/DARPA). The Government has certain rights in this invention.

### BACKGROUND OF THE INVENTION

1. Field of the Invention

The present invention generally relates to single reactant components immobilized over single electrodes for use in biosensors and methods of making and using thereof.

2. Description of the Related Art

Biosensor technology is the driving force in the development of biochips for the detection of gaseous pollutants, biological and chemical pollutants, pesticides, allergens, and microorganisms. A novel challenge is the development of effective biosensors based on fundamental research in biotechnology, genetics and information technology which will change the existing axiom of “detect-to-treat” to “detect-to-warn”.

Prior art methods for detecting environmental threats are primarily based on chemical, antibody, or nucleic acid based assays, which rely on chemical properties or molecular recognition to identify a particular analyte. See Paddle, B. M. (1996) Biosensors and Bioelectronics 11: 1079; Khaled, A., et al. (2003) Sensors and Actuators B. 94:103; and Yang, M., et al. (2003) J. of Micromechanics and Microengineering 13:864. These receptor/analyte binding/interaction assays are highly specific; however, the binding/interaction between receptors and analytes are often irreversible and thereby renders the biosensor useless for reuse. Additionally, the assays which rely on chemical properties or molecular recognition such as nucleic acid assays are environment specific as well as reaction specific, thus they are timely to conduct such that they are inadequate for use in early warning detection systems in the field. Furthermore, the prior art assays provide no functional information and they are unable to detect unknown or engineered analytes.

Cell based biosensors (CBBs) have been recently developed to overcome some of these shortcomings. CBBs have shown a promising future as cells have the capability to identify very minute concentrations of environmental analytes. These minute concentrations can be measured in parts per million (ppm) and in certain applications in parts per billion (ppb). The major drawback in the existing technology of CBBs is the improbable prospect of detecting all active analytes using a single type of cell or tissue physiologically. It is possible that particular analytes may undergo biotransformation, resulting in a secondary or tertiary compound of substantial physiological effect. Moreover, all the prior art CBBs rely on an array of cells and the communication between them. Also, the response and the sensitivity threshold of a single cell to a particular analyte have not yet been determined.

Currently, growth inhibitors are being used to control the cell population on the sensor surface creating an experimental control. This allows chemicals to be added to the cells and the long-term effect of the chemicals and be maintained and responses can be recorded. This effect on modifying the cell response to a stimulus for an extended duration of time, questions the validity of the existing technology. To achieve wide spread acceptance of the use of CBBs in field situations, noninvasive methods for determining the physiological status of cultured cells, methods of easier and more reliable data analysis, and methods of mass production and storage of cells used in CBBs are necessary. Unfortunately, these methods require highly skilled operators, sterile conditions, and unreliable source materials that are either impossible to achieve or unpractical in real life conditions.

Further, in order to develop effective, reliable and accurate CBBs that may be used as early detection and warning systems, changes or differences in the electrical activity of a single cell due to the presence or absence of a specific analyte in the environment external to the cell must be determined. Unfortunately, prior art methods do not provide the isolation of a single cell over a single electrode.

Thus, a need exists for single cells immobilized over single electrodes and methods of making and using thereof.

### SUMMARY OF THE INVENTION

The present invention generally relates to biosensors.

In some embodiments, the present invention provides a single reactant component immobilized over a single electrode. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

In some embodiments, the present invention provides a plurality of a single reactant component immobilized on a single electrode. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

In some embodiments, the present invention provides, a device comprising a single reactant component immobilized over a single electrode. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof. In some embodiments, the device further comprises a second single reactant component immobilized over a second single electrode. In some embodiments, the second single reactant component may be the same as or different from the single reactant component. In some embodiments, the device comprises a plurality of single reactant components immobilized over single electrodes, wherein the single reactant components may be the same or different. In some embodiments, the device further comprises a substrate upon which the single electrode is placed or immobilized. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof. In some embodiments, the device further comprises a permeation layer, an electrode pad, a measurement system, an environment chamber, a pulse generator, a micromanipulator, a CCD camera, a multichannel oscilloscope, a digital signal processor, a MEMS mixer, a suction system, a filter, a microreservoir, a microfluidic channel, a treatment cassette, a detection cassette, a data recording element, a reagent storage module, a mixing chamber, a reaction chamber, or combinations thereof

In some embodiments, the present invention provides a method of making the single reactant component immobilized over a single electrode immobilized over a single electrode, which comprises using an alternating current field to position the single reactant component over the single electrode. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof. In some embodiments, the method further comprises using AC electrical field to position single reactant component over the single electrode. In some embodiments, the method further comprises controlling the conductivity of a buffer solution which comprises the single reactant component.

In some embodiments, the present invention provides a biosensor which comprises a single reactant component immobilized over the single electrode. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

In some embodiments, the present invention provides a method of assaying, analyzing, or monitoring a target analyte which comprises contacting a sample suspected of having the target analyte with a single reactant component immobilized over a single electrode and detecting a change or a result, if any. In some embodiments, the result is compared with a standard or a control. In some embodiments, detecting the change comprises conducting AC impedance, impedance spectroscopy, cyclic voltammetry, AC voltammetry, pulse voltammetry, square wave voltammetry, AC voltammetry, hydrodynamic modulation voltammetry, conductance, potential step method, potentiometric measurement, amperometric measurement, current step method, Fourier transformation analysis, wavelet transformation analysis, or a combination thereof. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

In some embodiments, the present invention provides a method of identifying an unknown analyte as a known analyte or being similar to a known analyte which comprises contacting a sample suspected of having the unknown analyte with the single reactant component immobilized over a single electrode, determining a signature pattern vector for the unknown analyte and comparing the signature pattern vector with the signature pattern vector of the known analyte or the signature pattern vectors in a signature pattern vector database. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

In some embodiments, the present invention provides a signature pattern vector database comprising a plurality of signature pattern vectors for a plurality of reactant components immobilized over single electrodes. The single reactant component may be a chemical, a biomolecule, a microorganism, or a cell. In some embodiments, the chemical is a small molecule or a ligand. In some embodiments, the biomolecule is peptide, a protein, a nucleic acid molecule, or a receptor. In some embodiments, the microorganism is a bacterium. In some embodiments, the bacterium is E. coli. In some embodiments, the cell is an osteoblast, a glial cell, or a neuron. In some embodiments, the single electrode comprises iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof. In some embodiments, the single electrode has a diameter of about 60 μm to about 80 μm. In some embodiments, the single electrode has a diameter of about 40 μm to about 60 μm. In some embodiments, the single electrode has a diameter of about 20 μm to about 40 μm. In some embodiments, the single electrode is placed on or immobilized on a substrate. In some embodiments, the substrate comprises silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 is an optical micrograph of an electrode array having 25 platinum electrodes of 80 μm diameter and 200 μm center to center spacing in a 5×5 array. See Yang, M. et al. (2003) Sensors and Materials 15 (6):313, which is herein incorporated by reference.

FIG. 2A shows the positioning of single neuronal cell over a single electrode.

FIG. 2B shows the positioning of a single osteoblast over a single electrode.

FIG. 3 provides an FFT spectrum of osteoblast-ethanol.

FIG. 4 is an FFT spectrum of neuron response to ethanol at concentration of 9 ppm. Peaks at 314 Hz and 626 Hz represent the eigen values of the signature pattern vector.

FIG. 5 is an FFT spectrum of neuron response to hydrogen peroxide at concentration of 19 ppm. Peaks at 349 Hz and 853 Hz represent the eigen values of the signature pattern vector.

FIG. 6 is an FFT spectrum of neuron response to pyrethroid at concentration of 280 ppb. The peak at 514 Hz represents the eigen value of the signature pattern vector.

FIG. 7 is an FFT Spectrum of neuron response to EDTA at concentration of 180 ppm. Peaks at 227 Hz and 873 Hz represent the eigen value of the signature pattern vector.

FIG. 8 shows the distribution of the positive (PDEP) and negative (NDEP) dielectrophoretic forces on the chip surface.

FIG. 9 is a schematic representation of the measurement system which comprises extracellular electrophysiological measurement capabilities.

FIG. 10A shows a wavelet transformation analysis wherein the signal is filtered and the SPV is obtained.

FIG. 10B shows a wavelet transformation analysis where in local time domain, the response time is obtained.

FIG. 11 illustrates a process for making platinum patterned microarrays via lithography and wet etching.

FIG. 12 illustrates that photoluminescence of osteoblast sensing monitored.

FIG. 13 is a schematic representation of smart sensor of the present invention.

FIG. 14 is an electric circuit model for a unit area of the lipid bilayer membrane

FIG. 15A is a schematic representation of a synapse.

FIG. 15B is an electric circuit model for a synapse.

### DETAILED DESCRIPTION OF THE INVENTION

The present invention provides methods for isolating a single reactant component, such as a single cell, over a single electrode, methods for assaying the response or sensitivity of a reactant component immobilized over a single electrode to a second reactant component, methods for determining signature pattern vectors of reactant components, databases comprising signature pattern vectors, and biosensors comprising a single reactant component immobilized over a single electrode.

As used herein, members of a biological binding pair and biomolecules and cells that have a characteristic which may be observably modulated by a given compound are collectively referred to as “reactant components”. The reactant components according to the present invention may be natural or synthetic. The reactant components may be obtained from any organism.

Reactant components include probes and ligands known in the art that are used to detect other biomolecules, chemicals, microorganisms, cells, and the like. Some reactant components of the present invention include antigens that react with antibodies, oligonucleotides that hybridize to complimentary oligonucleotides, and ligands that bind receptors, and the like. In some embodiments, the reactant components are specific for a given microorganism. In some embodiments, the reactant component is a cell or a microorganism which produces an observable result in the presence of a given compound. For example, in some embodiments, the reactant component is a cell that produces a signature pattern in the presence of a given chemical as provided in the Examples herein.

Reactant components include biomolecules and compounds that bind to a target analyte (second reactant component) and may be used to probe for the presence of the target analyte. As used herein, “analyte” and “agent” are used interchangeably to refer to chemicals, biomolecules, cells, microorganisms, and the like, and include reactant components as defined herein. As will be appreciated by those in the art, the reactant component depends on the composition of the target analyte to be assayed. Various reactant components known in the art such as antigens, antibodies or fragments thereof (e.g. FAbs), small molecules, metal ions, chelators, proteins, peptides, enzymes, enzyme substrates, enzyme inhibitors, receptors, carbohydrates, nucleic acid molecules, aptomers, and the like may be used according to the present invention.

In some embodiments, the interaction or binding of one reactant component to its target analyte is specific. As used herein, “specific binding” or “specific interaction” between two reactant components mean that the reactant components bind or interact with each other with specificity sufficient to differentiate from the binding of or interaction with other components or contaminants in a given sample. It is possible, however, to detect reactant components without specificity using unique signature patterns as disclosed herein.

A. Single Cell Isolation

The present invention provides methods for isolating a single reactant component, such as a single cell of a given cell type, over a single electrode. Single reactant components may be immobilized over single electrodes by electrically assisted isolation, optical isolation, laser microbeam microdissection, and the like. See Meimberg, H., et al. (2003) Biotechniques 34 (6): 1238-1243; Umehara, S., et al. (2003) Biochemical and Biophysical Research Communications 305:534-540; Ozkan, M., et al. (2003) J. Biomedical Microdevices 5 (1):61-67; and Conia, J., et al. (1997) J. Clinical Laboratory Analysis 11:28-38, which are herein incorporated by reference. As exemplified herein, a single cell is immobilized over a single electrode utilizing alternating current fields based on the variance of in the dielectric properties of the cell.

Cell isolation by dielectrophoresis was developed to position single cells of various cell types over single electrodes. As used herein, “dielectrophoresis” refers to the lateral motion imparted on an uncharged particle as a result of polarization induced by a non-uniform electric field. Particle isolation by dielectrophresis may be extended to subcellular microorganisms, thereby allowing for the isolation of populations of microorganisms of the same biological state and viability. See Morgan, H., et al. (1999) Biophysical Journal 77:516-525, which is herein incorporated by reference. This enables the quantitative determination of the population of microorganisms required for inducing physiological responses in humans

The dielectrophoretic force acting on a spherical particle of radius, r, is given by the following Equation 1:
F_{DEP}=2πr³εₘRe(ƒ_{CM})∇E² Eq. 1
wherein
εₘ is the absolute permittivity of the suspending medium;
E is the local (rms) electric field;
∇ is the del vector operator; and
Re(ƒ_{CM}) is the real part of the polarization factor (Clausius-Mossotti factor),
defined by the following Equation 2:
   ƒ_{CM}=(εₚ*−εₘ*)/(εₚ*+2εₘ*) Eq. 2
   wherein εₚ* is the complex permittivity of the particle and εₘ* is the complex permittivity of the medium; and ε*=ε−jσ/ω wherein ε is the permittivity, σ is the conductivity, ω is the angular frequency of the applied field, and j=(−1)^{1/2}.

At crossover frequency, f_{crossover}, Equation 1 should be zero. Therefore, the crossover frequency is given by Equation 3 as follows:

$${f}_{crossover} = \frac{1}{2 π} \sqrt{\frac{( 2 {σ}_{m} + {σ}_{p} ) ( {σ}_{m} - {σ}_{p} )}{( 2 {ɛ}_{m} + {ɛ}_{p} ) ( {ɛ}_{m} - {ɛ}_{p} )}} \qquad Eq . 3$$

The dielectric properties of a biological shell can be characterized using a single shell model. See Markx, G. H., and Pethig, R. (1995) Biotech. Bioeng. 45:337-343; and Huang, Y., et al. (2002) Anal. Chem. 74:3362-3371, which are herein incorporated by reference. The single shell model regards the cell as a homogeneous, high conductivity aqueous interior surrounded by a poorly conducting plasma membrane, i.e. the shell. The dielectric permittivity, ε, and the conductivity, σ, at the interior and the shell are assumed to be frequency independent over the whole frequency range of study (less than about 10 MHz). However, the dielectric properties of the cell, εₚ and σₚ, are frequency dependent on the interface between the interior of the cell and the membrane shell. This is indicated in Equation 4 as follows:

$${ɛ}_{p}^{*} = {ɛ}_{m}^{*} \frac{{( \frac{r + d}{r} )}^{3} + 2 \frac{{ɛ}_{int}^{*} - {ɛ}_{mem}^{*}}{{ɛ}_{int}^{*} + 2 {ɛ}_{mem}^{*}}}{{( \frac{r + d}{r} )}^{3} + \frac{{ɛ}_{int}^{*} - {ɛ}_{mem}^{*}}{{ɛ}_{int}^{*} + 2 {ɛ}_{mem}^{*}}} \qquad Eq . 4$$

wherein
d is the thickness of the plasma membrane;
εᵢₙₜ* is the complex permittivity of interior; and
εₘₑₘ* is the complex permittivity of plasma membrane.

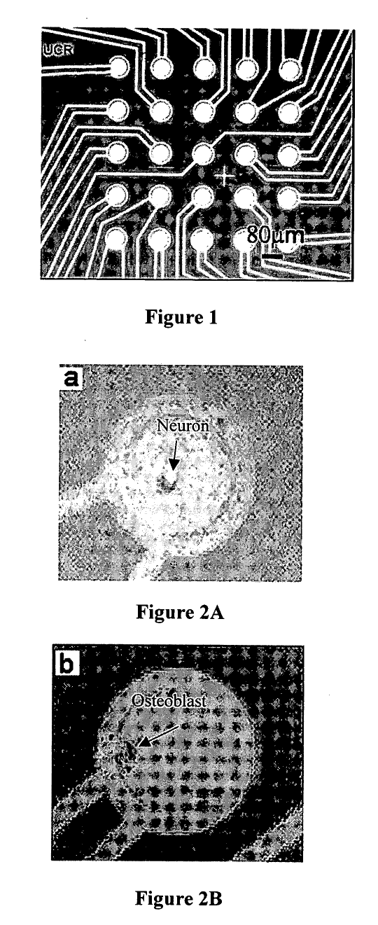

The crossover frequency, f_{crossover}, is the frequency at which the cells experience a zero dielectrophoretic force based on the geometry and design of the electrodes a non-uniform AC field was set up. The AC voltage was applied to the electrodes through the micromanipulators. The voltages were transmitted from the electrode pads to the electrodes through the electrode leads. The non-symmetrical arrangement of the electrode leads and a slight skew in the electrode arrangement as shown in FIG. 1 are the reasons for obtaining the gradient electric field. The parameters that determine the location of cells on a microelectrode array on a substrate are the dielectric properties of the cells, the conductivity of the medium, applied AC frequency and the peak-to-peak voltage.

When the conductivity of the medium is less than that of the cells, then the cells experience positive dielectrophoretic (positive DEP) force, similarly when the conductivity of the medium is greater than that of the cells, the cells experience negative dielectrophoretic (negative DEP) force. For a certain medium conductivity, frequency can be tuned and the cell can be made to experience both positive and negative DEP forces. The frequency at which the cell does not experience any dielectrophoretic force is known as crossover frequency. Table 1 gives the separation buffer for obtaining positive and negative DEP forces for particular cell types and their associated conductivity, the frequencies at which the cell types experience positive DEP force, negative DEP force, the crossover frequency, and the applied peak to peak voltage.

TABLE 1
Parameters for DEP for Different Cell Types

| | | Conductivity of | Positive | | Negative | | Cross | | |
|---|---|---|---|---|---|---|---|---|---|
| | Separation buffer | buffer solution | DEP | | DEP | | over | | Vₚₚ |
| Cell Type | for DEP | (mS/cm) | frequency | | frequency | | frequency | | (Volts) |
| Neuron | 250 mM | 1.2 | 4.6 | MHz | 300 | kHz | 500 | kHz | 8 |
| | Sucrose/1640 | | | | | | | | |
| | RPMI | | | | | | | | |
| Osteoblasts | Phosphate Buffer | 4.09 | 136 | kHz | 25.5 | kHz | 65 | kHz | 8 |
| | Saline/250 mM | | | | | | | | |
| | Sucrose | | | | | | | | |

In order to obtain a particular cell type to be positioned over electrodes, the cells must experience positive DEP force. The cells experiencing positive DEP force are positioned at the electrode edges, which are the regions of high electric field. The glial cells experiencing negative DEP force are positioned at the areas between the electrodes, which are the low electric field regions. The ideal conductivity of the separation buffer in an iterative manner was determined. As provided in Example 1, neurons were separated from glial cells of a neuronal cell culture and later positioned on the electrodes using an alternating current field. 25 μl of the nonhomogeneous culture comprising of neurons and glial cells was injected onto the microelectrode sensing array using the microfluidic inlet/outlet system. The culture was pumped into the positioning/sensing area at a rate of about 20 μl per minute. The gradient electric fields were applied to the electrodes that function as both the positioning as well as the sensing sites. The parameters applied are indicated in Table 1.

Based on the above mentioned parameters, single cell arrays of neurons/glial cells were formed. The sensing area was then washed with Dulbecco's Modified Eagle's (DME) medium at a flow rate of about 10 μl per minute in the presence of the electric field to remove the nonessential and superfluous cells that may compromise the accuracy of the sensor. The electric field was then switched off and the chemical analyte mixture garnered from the atmosphere was injected onto the sensing area. The known/unknown chemical analytes were sensed and the signals were transduced by the cell membrane. The signal was then read-out using probes. FIG. 2A shows the positioning of single neuronal cell over a single electrode. FIG. 2B shows the positioning of a single glial cell over a single electrode. The subcellular particles, E. coli, were positioned in a similar manner. The E. coli have a negative surface charge. In a buffer of 1% peptone having conductivity of about 515 μS/cm E. coli are positioned onto the electrodes that function as sensing sites at a peak-to-peak voltage of about 1V and about 14.7 kHz. The population of E. coli positioned at about 10×10⁻⁷ concentration in 1 ml are identified as the detection limit in detect-to-warn biosensing applications.

The parameters that determine the relative polarizability are applied frequency, conductivity, and the relative permittivity of the buffer solution. It was found that single neuron positioning was achieved at about 4.6 MHz, with a peak to peak voltage of about 8V, and with a buffer solution conductivity of about 1.2 mS/cm. These parameters may be readily determined in a similar manner for other animal and plant cells.

B. Response Or Sensitivity of Single Reactant Component

The present invention also provides methods of detecting, monitoring, measuring, or analyzing a response or sensitivity of a reactant component, such as a single cell, to a variety of extracellular analytes.

The method for isolating a single reactant component over a single electrode as provided herein allows one to detect, measure, analyze, and assay any electrical responses or sensitivities a given reactant component, such as a given cell type, has to a variety of extracellular analytes. Thus, a single reactant component, such as a single cell, immobilized over a single electrode may be used to perform in situ assays for a variety of applications where minute changes in the concentration of an analyte contacted with the reactant component modifies or modulates at least one chemical, physical, or electrical characteristic of the reactant component.

The experiments in Example 2 were conducted on mammalian cells, rat osteoblasts and neurons (18 day embryonic Wistar rat) having highly excitable cell membranes. As provided herein, the responses of single cells of rat osteoblasts and neurons were recorded and analyzed for the following agents: ethanol, hydrogen peroxide, ethylenediaminetetraacetic acid (EDTA), and pyrethroids, including syntethtic pyrethrum such as bifenthrin. A unique response of each cell type to each agent was determined. The cell type with maximum sensitivity and shortest response time was determined from comparative studies provided in Example 3. Here, two out of four agents are discussed. Additionally, the response of each cell type to a mixture of the four agents was determined in order to simulate the real time field conditions. The results obtained for neurons are discussed.

Ethanol, hydrogen peroxide, ethylenediaminetetraacetic acid (EDTA), and pyrethroids were selected for analysis for the following reasons:

Ethanol produces anesthetic effects but in a milder form as compared to pentobarbitone and ketamine, though the mechanism of action is assumed to be substantially the same. See Singh, J. et al. (2000) Ind. J. Pharmacology 32:206-209, which is herein incorporated by reference. The determination of single cell ethanol sensitivity will help identify the lowest threshold concentrations for analytes whose physiological response mechanisms mimic that of ethanol. Thus, the biosensors of the present invention may be used to assay anesthetics or identify new anesthetics.

Hydrogen peroxide is a neuroactive compound that is water-soluble and affects or modulates the electrophysiological mechanisms of nerve cells. Hydrogen peroxide is one of the major metabolically active oxidants present in plants and animals. Hydrogen peroxide leads to apoptosis as well as cellular degradation. The effect of hydrogen peroxide on living organisms in vivo is similar to the behavioral responses obtained from exposure to carcinogenic chemicals, such as rotenone. See SENSORS: A COMPREHENSIVE SURVEY (1995) ed. W. Gopel, J. L Hesse and J. N. Zemel, Trends in Sensor Technology/Sensor Markets 295-336, which is herein incorporated by reference. Therefore, the determination of single cell sensitivity to hydrogen peroxide may be used to assay or identify analytes and concentration of analytes that cause cancer, abnormal cell proliferation, apoptosis, or cellular degradation.

Pyrethroids are a large family of chemicals that is divided into four generations. The first generation consists of only one pyrethroid, namely allethrin; the second, third, and fourth generations comprise derivatives from the first generation and are active ingredients in most commercial pesticides. Pyrethroids share similar modes of action, resembling that of DDT, and are considered axonic poisons (i.e. chemicals that cause the paralysis and degradation of the fundamental signal transducing conduit in a neuron such as the axon-like pyrethroids and avermectins). Therefore, the determination of single cell sensitivity to pyrethroids may be used to identify analytes and concentration of analytes that have DDT like activity or are axonic poisons. Pyrethroids are expected to produce a “knock down” effect, the loss of coordination among individual cells at a cellular level that results in the phase shift of signals that are transmitted via the axons of the cells which leads to the loss of coordination of a microorganism.

Ethylenediaminetetraacetic acid (EDTA) belongs to a class of synthetic, phosphate-alternative compounds that are not readily biodegradable and once introduced into the general (ambient atmospheric) environment can redissolve toxic heavy metals. EDTA and similar chemicals act as stores for toxic heavy metals due to their ability to redissolve these substances.

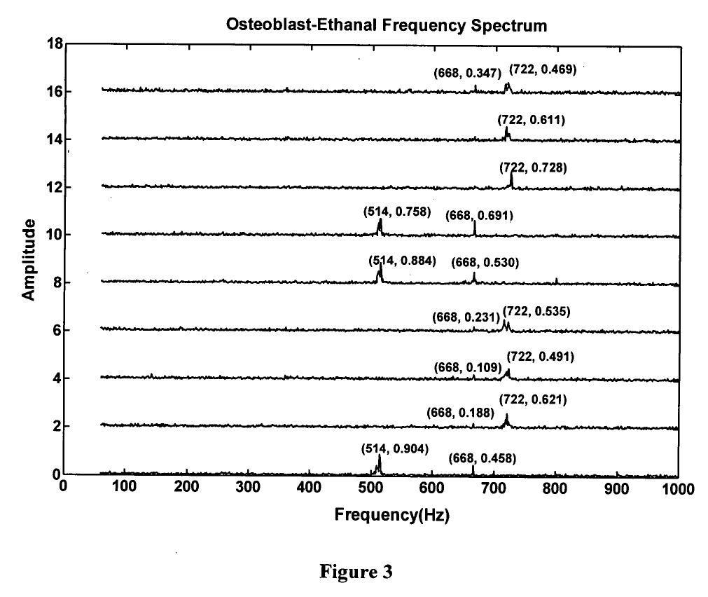

FIG. 3 provides an FFT spectrum of osteopath-ethanol, a characterization of cell signal both in time domain and frequency domain.

C. Signature Pattern Vectors

The method for isolating a single reactant component over a single electrode as provided herein allows the elucidation of characteristic signature pattern vectors (SPVs) of the reactant component for a given analyte or analytes substantially similar to the given analyte. SPVs of various cell types allows one to determine the effect of a broad spectrum of analytes ranging from highly toxic and physiologically damaging to relatively less toxic so as to determine and evaluate the time window of response of a particular cell type for a specific known analyte based on varying concentrations. Predetermined SPVs allows the identification of an unknown analyte that may exhibit a certain bioactivity by comparing the SPV of a given cell type to the unknown analyte with a library comprising at least one SPVs of the given cell type to a known analyte.

As provided herein, experiments were conducted based on the hypothesis that a unique SPV would be generated for each cell type for a specific chemical as different chemicals bind to different ion channel receptors for different periods on the cell membrane thereby modifying the electrical response of the cell in a unique manner. The results provided herein support the hypothesis that each cell type has a unique SPV for a specific analyte. Additionally, the results show that a cell maintains its unique SPV to a specific analyte when a combination of analytes are contacted with the cell in a cascaded manner.

Ion channels are membrane proteins that control cell permeability to specific ions. Ion channels are responsible for signal generation and transmission in the cell. The type, properties, number, and specific location of ion channels determine the signaling properties of cells, such as neurons, and the regulation of ion channel activity contributes to the bursting process. Ca²⁺-activated K⁺ channels that are responsible for the bursting frequency are observed in the fast FFT spectra. These channels are activated by sub-micromole concentrations of intracellular Ca²⁺ and generate after-hyper-polarizations (AHP) following single or multiple action potentials. Hence the production of the after hyperpolarization potentials stabilizes the frequency of firing of a neuron to certain non-periodic peaks namely 220 Hz, 315 Hz, 450 Hz, 550 Hz, 750 Hz and 860 Hz. AHP limit the number of action potentials and slow down the firing frequency of neurons during sustained stimulations, a phenomenon known as “spike frequency adaptation”. See Stocker, M. et al. (1999) PNAS 96:4662-4667, which is herein incorporated by reference. The frequency spikes observed in the response pattern can be attributed to this effect.

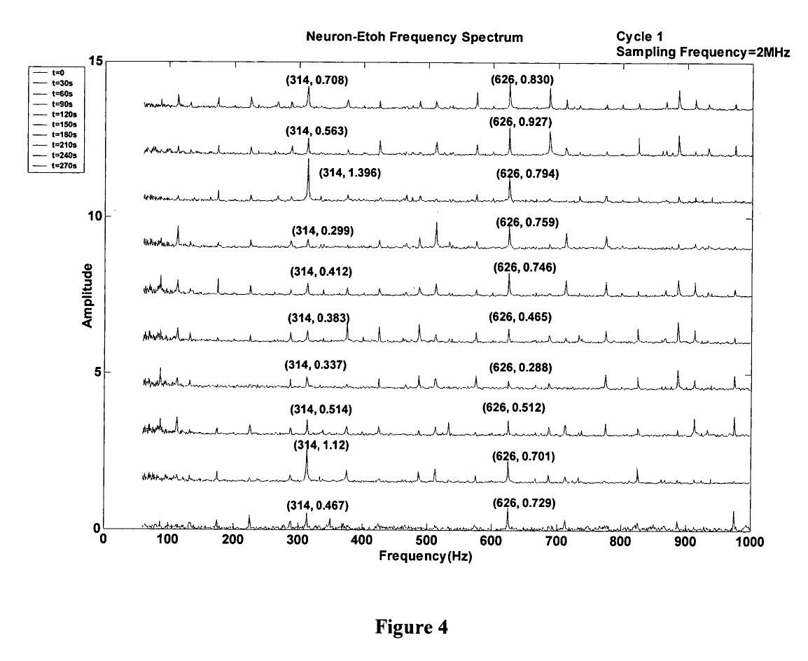

Glycine receptor/channels (GlyR) are sensitive to pharmacologically relevant concentrations of ethanol. Since glycine has inhibitory effects on neuronal activity, potential of GlyR function would be expected to enhance neuronal inhibition and perhaps contribute to the neuronal depressant effects of ethanol. This would lead to a long hyper-polarization period of the neurons, which would result in the increase in the intracellular Ca²⁺ levels. This will result in a low frequency of firing (315 Hz). With the dissipation of ethanol throughout the cell the bursting rate increases (627 Hz) which is consistent with the observed values. See Ye, J. H. et al. (1999) J. Pharmacol. Exp. Ther. 290:104-111, which is herein incorporated by reference. In addition, when the hippocampal neurons are excited by ethanol, they are potentiated by serotonin and by serotonergic drugs acting at serotonin 5HT2 type receptors. See Brodie, M. S. et al. (1995) J. Pharmacol. Exp. Ther. 273 (3):1139-1146, which is herein incorporated by reference. As shown in FIG. 4, this results in creating two modes for firing of the neurons the low frequency mode of eigen values, the unique frequency states at which the modified electrical response peaks, of the SPV, 314 Hz and the high frequency mode of 626 Hz.

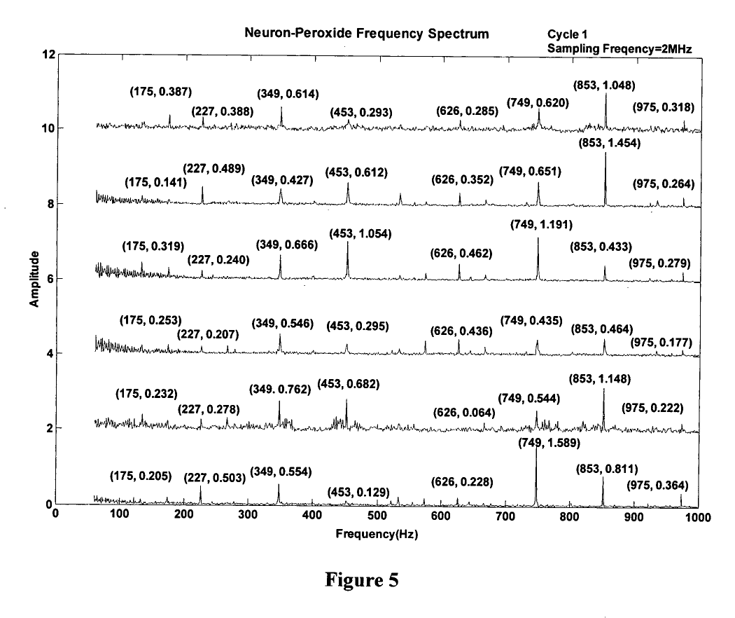

Addition of hydrogen peroxide to a neuron results in a positive feedback loop, which is evidenced by the expression of excitotoxicity and oxidative stress. See Doble, A. et al. (1998) Trends in Pharmacological Sciences 19:9-11, which is herein incorporated by reference. Excessive amounts of glutamate, or prolonged action of glutamate, at receptors result in excessive Ca²⁺ influx, via voltage-dependent C²⁺ channels or via glutamate receptor-linked channels that allow C²⁺ influx such as the N-methyl-D-aspartate channel, resulting in prolonged periods of elevated intracellular Ca²⁺. Such elevated Ca²⁺ can activate Ca²⁺-dependent enzymes such as phospholipase A2 that can release arachidonic acid whose metabolism generates superoxide anion. Mitochondria Ca²⁺ cycling also results in mitochondrial damage increasing the production of superoxide anion. The elevated Ca²⁺ also stimulates release of more glutamate. As shown in FIG. 5, this is indicated by the high frequency of firing of the neurons (349 Hz and 853 Hz) within 30 seconds of addition of hydrogen peroxide.

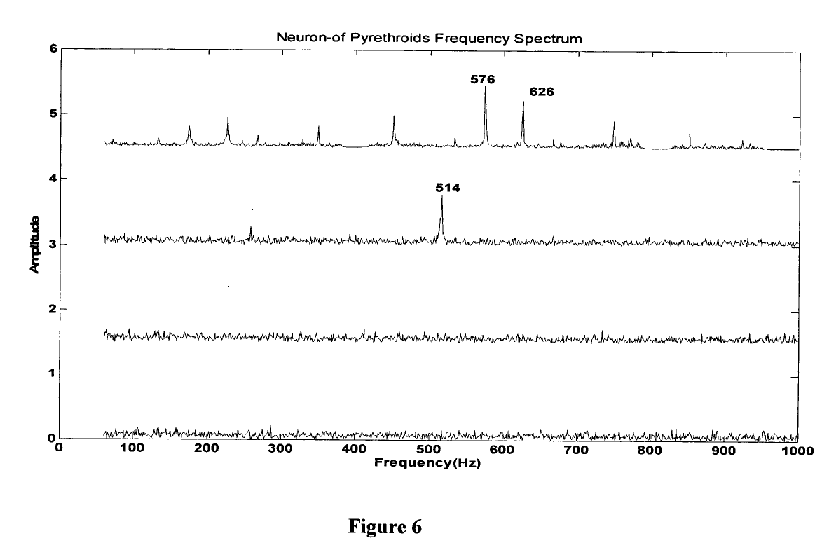

Pyrethroids share similar modes of action, resembling that of DDT, and are considered axonic poisons. Pyrethroids work by keeping open the sodium channels in neuronal membranes. There are two types of pyrethroids. Type I, which includes tralomethrin, among other physiological responses, have a negative temperature coefficient, resembling that of DDT. Type II, which includes d-trans allethrin in contrast have a positive temperature coefficient, showing increased apoptosis with an increase in ambient temperature. Pyrethroids initially stimulate neurons to produce repetitive discharges and eventually cause paralysis. Such effects are caused by their action on the sodium channel, the stimulating effect of pyrethroids is much more pronounced than that of DDT. The action of the pyrethroid on Na⁺ channel causes an increase in the depolarization due to after potential which causes an increase in the rate of firing due to an increased influx of Ca²⁺ ions, and localization of firing at a higher frequency value (576 Hz and 626 Hz) shown in FIG. 6. The eigen value of firing in this case is at 514 Hz, the stabilization of the frequency at a lower value is due to a slight hyper-polarization due to the delayed effect of the pyrethroid on the chloride channels. The final stage of action of the pyrethroid before apoptosis is the generation of repetitive after discharges, which can be observed from the SPV of the neuron for pyrethroids. See Lund, A and K. Narahashi (1983) Environmental Neurotoxicology 12:167-186, which is herein incorporated by reference.

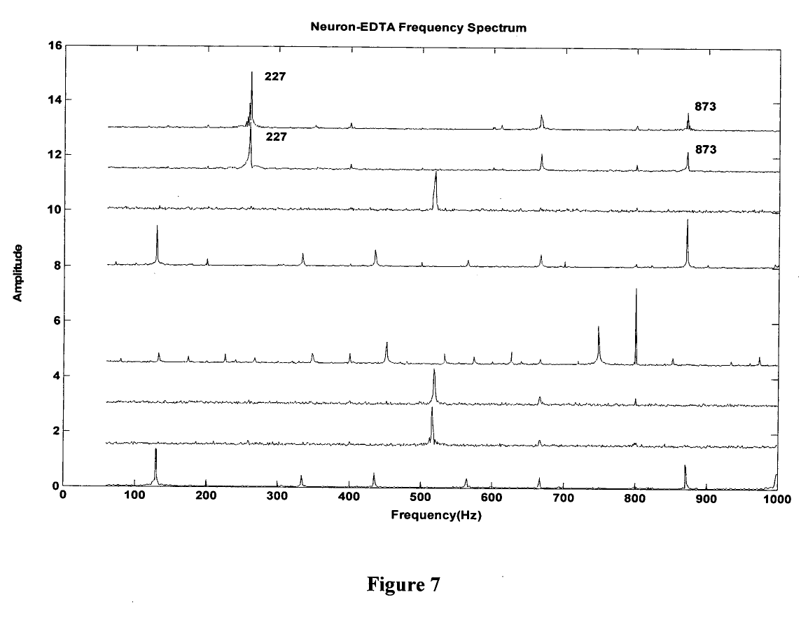

EDTA is a chemical that tightly binds Ca²⁺. When a neuron is stimulated in the presence of EDTA, it fires an action potential, but transmission of the action potential never occurs. This is due to an inflow of Ca²⁺ ions into the cell via the cell membranes. This is necessary for the release of GABA and NMDA neurotransmitters essential in cell communication. See Millecamps, S. et al. (1999) Nat. Biotech. 17 (9):865-869, which is herein incorporated by reference. Hence, the initial firing rate of the neuron is low (227 Hz). As shown in FIG. 7, further action of EDTA results in cell paralysis that results in a sudden increase in firing rate (873 Hz).

D. SPV Database

The present invention also provides a database comprising at least one SPV of at least one reactant component for at least one analyte. To create a SPV database according to the present invention, the response in the frequency domain of a reactant component, such as a specific cell type, to a specific analyte is first determined. Then the SPV is determined for stepwise decremented concentrations for analytes, such as specific chemical/biological analytes. The response in the frequency domain of the reactant component for a cascaded application of the analytes is determined. The response in the frequency domain for a specific reactant component for a fluid sample containing a mixture of analytes may be determined in order to simulate real field conditions. Last, the SPVs that indicate the unique response pattern in the frequency domain for each reactant component for various analytes is complied into a database. The database may be a searchable electronic database.

E. Unknown Analyte Identification

The present invention also provides methods for identifying or classifying at least one unknown analyte based on a SPV for a given reactant component to at least one known analyte. Generally, the methods comprise comparing the SPV of the unknown analyte to the SPV of a known analyte or an SPV database.

F. Calcium Imaging

The SPVs obtained herein are related in a direct or an indirect manner to a variation in the intracellular calcium levels. To verify the physiological changes responsible for the unique behavior in the neurons based on the specific chemical analytes calcium imaging and immunohistochemical staining as provided in Example 4 may be used as well as other methods known in the art.

G. Sensors

The present invention provides accurate, sensitive, fast, inexpensive, portable, easy to use, and disposable or reusable biosensors that can be used to detect, measure, and monitor the presence of a variety of analytes in fluid and aerosol samples such as the air and water.

The method for isolating a single reactant component, such as a single cell of a given cell type, over a single electrode according to the present invention may be used to pattern a plurality of reactant components on a microelectrode array. The single reactant component microelectrode array may be integrated into a microsensor such as a biosensor, e.g. associated with a monitor and a microfluidic system for use as a “detect-to-warn” sensor.

The biosensors of the present invention include cell-based-biosensors (CBBs) and protein-based-biosensors (PBBs). CBBs comprise at least one single cell immobilized over a single electrode or a plurality of single cells of the same or various cell types over a plurality of electrodes. PBBs are developed from the information obtained from single cell/single electrode studies.

A CBB of the present invention comprises a microelectrode array, such as a 4×4 or 5×5 microelectrode array known in the art, on a substrate such as glass, silicon, titanium, quartz, and the like. In some embodiments, the electrodes are about 30 μm in diameter, with about 100 μm center to center spacing. Each electrode is connected to two separate electrode pads through electrode leads.

As used herein, the term “array” refers to an ordered spatial arrangement, particularly an arrangement of molecules, such as biomolecules including the binding molecules or probes or microelectrodes as described herein. In some embodiments, the arrays of the present invention comprise a matrix of addressable locations. As used herein, the term “addressable array” refers to an array wherein the individual reactant components have precisely defined x and y coordinates, so that a given reactant component at a particular position in the array may be identified, monitored, evaluated, and the like. As used herein, the terms “bioarray”, “biochip” and “biochip array” refer to an ordered spatial arrangement of biomolecules on a microelectrode array on a substrate. Biomolecules include nucleic acids, oligonucleotides, peptides, proteins, ligands, antibodies, antigens, and the like. In some embodiments, the bioarrays of the present invention comprise a reactant component, such as a member of a biological binding pair, biomolecules or cells that have a characteristic which may be observably modulated by a given compound.

The microelectrode arrays of the present invention need not be in any specific shape, that is, the electrodes need not be in a square matrix shape. Contemplated electrode array geometries include: squares; rectangles; rectilinear and hexagonal grid arrays with any sort of polygon boundary; concentric circle grid geometries wherein the electrodes form concentric circles about a common center, and which may be bounded by an arbitrary polygon; and fractal grid array geometries having electrodes with the same or different diameters. Interlaced electrodes may also be used in accordance with the present invention. In some embodiments, the array of electrodes comprises about 9 to about 16 electrodes in an at least about a 4×4 matrix. In some embodiments, the array of electrodes comprises about 16 to about 25 electrodes in an at least about a 5×5 matrix. Other sized arrays known in the art may be used in accordance with the present invention.

In some embodiments, the electrodes in the microelectrode arrays of the present invention range in diameter from about 20 μm to about 80 μm . The microelectrode arrays of the present invention may have a distance of about 100 μm to about 200 μm from center to center of the electrodes, regardless of the electrode diameter. For example, in some embodiments, the distance between the centers of two neighboring electrodes is about 100 μm.

The electrodes may be of various shapes and sizes known in the art and may be or may not be flush with the surface of the substrate. The electrodes of the present invention may comprise noble metals such as iridium and/or platinum, and other metals, such as, palladium, gold, silver, copper, mercury, nickel, zinc, titanium, tungsten, aluminum, and the like, as well as alloys of various metals, and other conducting materials, such as, carbon, including glassy carbon, reticulated vitreous carbon, basal plane graphite, edge plane graphite, graphite, and the like. Doped oxides such as indium tin oxide, and semiconductors such as silicon oxide and gallium arsenide are also contemplated. Additionally, the electrodes may comprise conducting polymers, metal doped polymers, conducting ceramics, conducting clays, and the like.

In some embodiments of the present invention, one or more of the electrodes may be proximate to a “getter” structure. The “getter” structure may comprise a second electrode which may be of any shape, size and material discussed above. In some embodiments, the getter structure scavenges electrochemically generated reagents alone or in conjunction with a scavenging solution and/or a buffering solution or reduces or eliminates the diffusion of ions into nearby electric sources such as semiconductor circuitry.

An electrode used in accordance with an array of the present invention may be connected to an electric source using methods known in the art including CMOS switching circuitry, radio and microwave frequency addressable switches, light addressable switches, and direct connections from an electrode to a bond pad on the perimeter of a substrate.

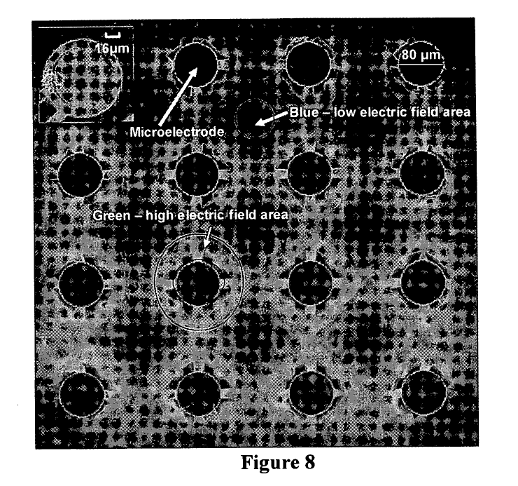

FIG. 8 shows the distribution of the positive (PDEP) and negative (NDEP) dielectrophoretic forces in a microelectrode array on a substrate.

As provided in Example 3, recent studies of the CBBs of the present invention provide that less than about 0.2 seconds of response time can be achieved from live cells with the chemical concentrations in the ppm and even in ppb range. No false positives were observed from the repeated experiments. The cell types of the CBBs of the present invention may selected to provide faster response times for given chemical toxins as the recent studies also provide that different cell types provide different response times. Table 2 depicts some of the preliminary results of response times to certain chemicals at given low concentrations using different types of cells. These experiments were successfully repeated several times.

TABLE 2
Response Time of Osteoblast and Neuron Cells
to EDTA, Ethanol, Peroxide, and Pyrethroids

| | EDTA | Ethanol | Peroxide | Pyrethroids |
|---|---|---|---|---|
| Osteoblast | 0.14 sec per | 0.71 sec per | 0.42 sec per | 0.53 sec per |
| | 280 ppm | 190 ppm | 25 ppm | 890 ppm |
| Neuron | 0.28 sec per | 0.19 sec per | 0.06 sec per | 0.23 sec per |
| | 180 ppm | 9 ppm | 19 ppm | 280 ppb |

Microelectrode devices known in the art may be used according to the present invention. For example, for the experiments described herein, an experimental platform comprising a 5×5 electrode array on a substrate, a silicone chamber, and a measurement system was used. The micrograph of the fabricated electrode array on a substrate is shown in FIG. 1 with a 5×5 platinum electrode array pattern, electrode diameter of 80 μm and with a 200 μm center to center spacing. Two platinum plated leads (6 μm, thick) from each electrode terminated at two separate electrode pads. The dimensions of the electrode pads were 100 μm×120 μm. The electrode array on the substrate was coated with a permeation polyethylamine (PEI) and collagen with a volume of 200 μl (PEI, 1 mg/ml, Collagen 1X, Sigma, St. Louis, Mo.). The electrode array on the substrate with the aqueous coating of the permeation layer was incubated for about 12 hours prior to the experiment at 37 ° C. and 5% CO₂. The purpose of the permeation layer is to improve the cell adhesiveness to the microelectrode array.

The microelectrode array on the substrate was covered with an environment chamber, such as a silicone chamber (16 mm×16 mm×2.5 mm). The silicone chamber had an opening at the center (1.2 mm×1.2 mm×2.5 mm). The silicone chamber was covered by a glass cover slip (500 μm thick). The volume of the silicon chamber was 25 μl. The silicone chamber and the glass cover slip were used to maintain a constant local environment for the cell culture in order to obtain reliable measurements.

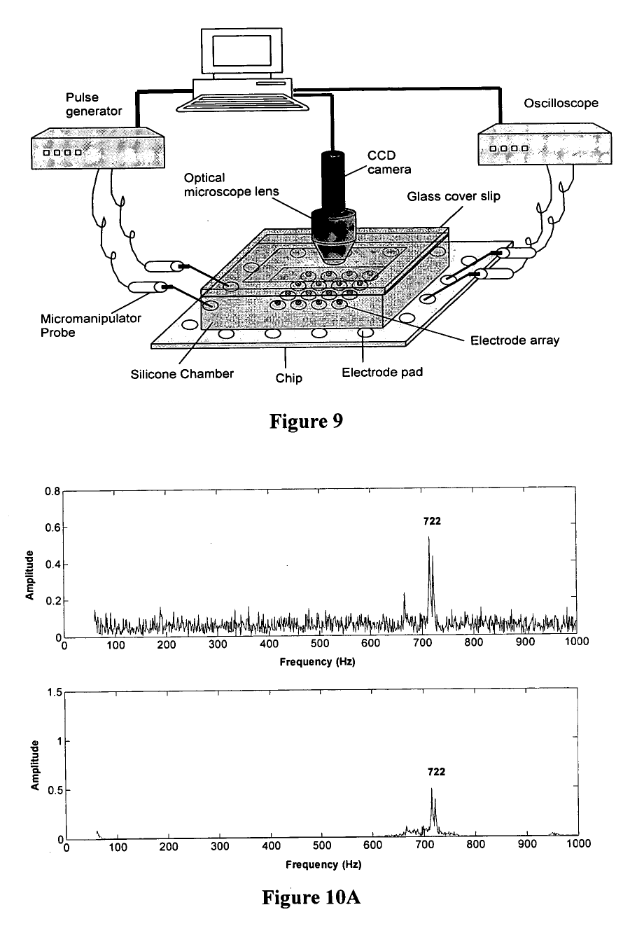

FIG. 9 shows a schematic representation of the measurement system which comprises extracellular positioning, stimulating and recording units. The cells were separated and positioned over the electrodes by setting up a gradient AC field using an extracellular positioning system, comprising of a pulse generator and micromanipulators. The AC signal from the pulse generator was fed to the electrode pads of the selected electrodes using the micromanipulators. The extracellular recordings from the cells were amplified and recorded on an oscilloscope. The supply and measurement systems are integrated using general-purpose interface bus (GPIB) control and controlled through LabVIEW (National Instruments, Austin, Tex.).

The cell cultures were monitored using an inverted microscope. The cell separation, positioning, and network formation were imaged using an optical probe station under 8× and 25× and 50× magnification. The network growth was optically monitored using an upright microscope equipped with a CCD camera. The signals obtained from the cells during positioning and network formation were analyzed using MATLAB® (Mathworks, Natick, Mass.).

Then fast Fourier transformation (FFT) and wavelet transformation (WT) analysis were used to extract important information from the extracellular action potential (AP) for the neurons. Specific spectrum power vectors (SSPVs) and the relative spectrum power values (RSPVS) were found for osteoblast. Quantitative dose response curves and response times were also obtained for the single cell systems.

Using fast Fourier transformation (FFT) analysis, the shifts in the signal's power spectrum were analyzed. The ionic channels modulated by the analyte may be classified. The broad-spectrum sensitivity of CBBs offers the capability for detecting previously unknown biological analytes. Changes in the extracellular AP shape may be used to monitor the cellular response to the action of an analyte such as pharmaceuticals and toxins. Power spectral density analysis may be used to classify the action of a biologically active analyte. The power spectral density may be approximated by examining the rms power in different frequency bands.

However, FFT analysis is the transformation based on the whole scale, i.e. either absolutely in time domain, or absolutely in frequency domain. Thus, it is impossible to express the local information in time domain. Therefore, wavelet transformation (WT) analysis was used to extract the information from the local time domain. WT is the time-scale (time-frequency) analysis method with the characterization of multi-resolution analysis, which can express the local characterization of signal both in time domain and frequency domain and can be used to extract important information from the extracellular action potential (AP), such as the response time analysis and concentration analysis, the amplitude modulation (AM).

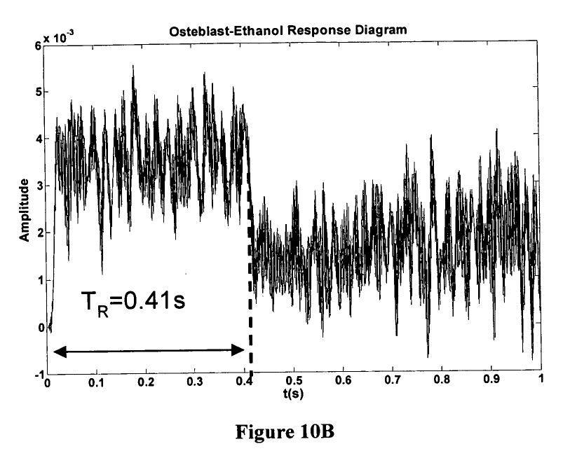

FIG. 10A shows a wavelet transformation analysis wherein the WT is used to filter the signal, and then the frequency pattern for certain chemical agent is obtained, and FIG. 10B shows, in local time domain, the response time.

CBBs of the present invention may be reused as the single cells immobilized over the single electrons respond to specific chemicals at very low concentrations, such as ppb, and then regain their initial characteristics after a short period of time, such as about ten minutes. The single cells may then be exposed to the same chemical or a different chemical.

A PBB of the present invention is capable of detecting, monitoring, measuring, or assaying chemical or biological analytes, such as chemical and biological warfare analytes and analytes associated with environmental threats, based on the interaction between at least one protein patterned on a microarray platform and at least one analyte in a fluid sample.

The PBB of the present invention may be developed by extracting at least one receptor protein associated with modifying or modulating a given characteristic, such as a chemical, physical, or electrical response, of a single cell immobilized over a single electrode and micropatterning the receptor on a substrate such as silicon/silicon nitride, quartz, titanium, ceramic, plastic, and the like. The proteins are patterned into electrode arrays in the form of self-assembled monolayers (SAMs). The design of the protein patterns is specific to the given application which may be readily determined by one skilled in the art. Generally, using methods known in the art, the protein pattern is determined and then that specific area is masked. Then electrode leads of desired geometry are fabricated using deposition techniques known in the art. The proteins are then imprinted onto the chip surface using microprinting, elastomeric stamps, or a variety of other methods known in the art.

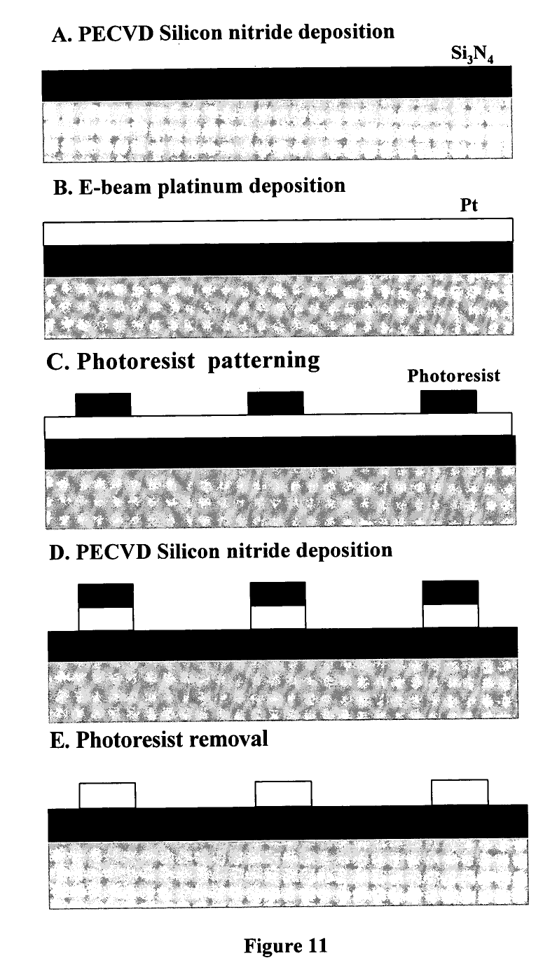

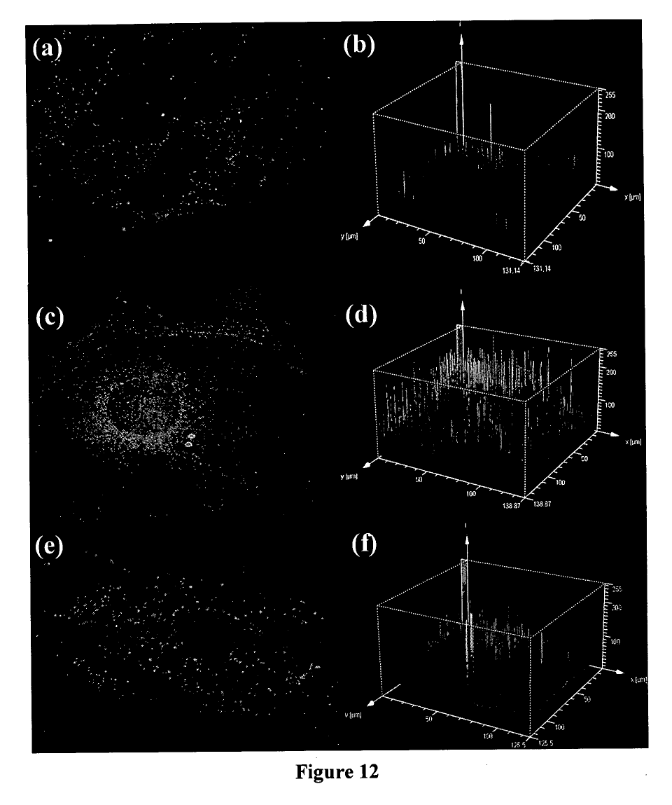

For example, platinum patterned microarrays may be fabricated as shown by the process sequence of FIG. 11, a one step lithography and wet etching process, to make an array of electrodes. Then a soft elastomeric microstamp may be fabricated using a polydimethylsiloxane (PDMS) molding process, as shown in FIG. 12. Generally, an inverse layout of the patterns are generated using a thick negative photoresist, such as SU-8 (MicroChem Inc, Newton, Mass.) over a silicon substrate. Then PDMS molding is carried out to create a soft stamping layer. The soft stamp may be used to imprint a layer of the protein onto the electrode array as shown in FIG. 11.

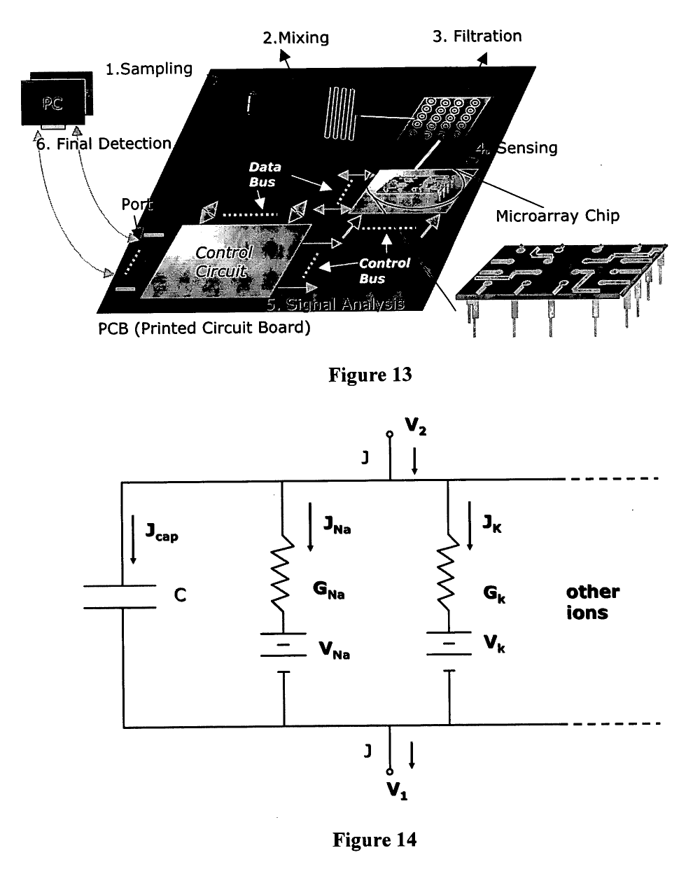

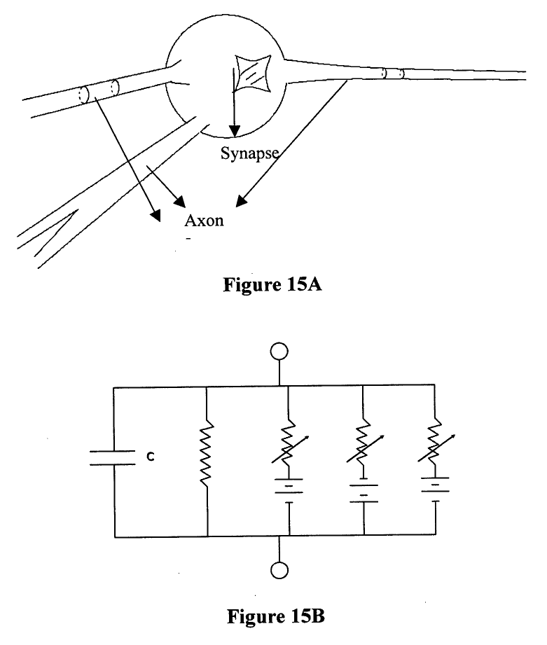

The electrical activity obtained from the PBB is analyzed and compared with an existing database for determining the presence of at least one analyte or a combination of analytes. The control and data acquisition circuitry may be integrated on a printed circuit board (PCB). The analysis software may be developed to decode the electrical activity from the PBB in order to determine within about 2 seconds or less the type of analyte present in the fluid sample. FIG. 13 shows a PBB of the present invention. An advantage of the present invention is that it does not rely on cell behavior, which varies from assay to assay, but on the analyte/receptor interaction.

In an alternative embodiment, instead of monitoring the electrical responses of the cell, a change in the photoluminescence of a porous silicon layer due to the receptor/analyte interaction may be monitored as shown in FIG. 14. For example, a layer of porous silicon may be fabricated by electrochemical etching of silicon on silicon-on-insulator (SOI) or silicon-on-sapphire (SOS) substrate using methods known in the art. The isolated receptor can be embedded into the pores of the porous silicon layer which will serve as the sensing medium. Chemical analyte binding to the protein will change the refractive index of the photoluminescence which change may be detected, monitored, measured, or assayed.

For both CBBs and PBBs, a microelectrode array may be hermetically sealed with a chamber, such as a silica rubber chamber, which encloses a buffer solution containing single cells or receptors of interest as shown in FIG. 11. Other materials and designs known in the art may be used. The electrical activity of the individual cells is preferably recorded continuously using associated electronic circuitry. The electrical signals may be read out using a multichannel oscilloscope and the data obtained may be analyzed using a digital signal processing method known in the art. An example of a monitoring or measuring system is shown in FIG. 12. The fluid sample to be tested may be introduced into the system via an opening or a window. The concentration of the fluid sample is stepwise diluted. The dilutions may be serial dilutions. Then the speed of response and the activity pattern for each dilution is recorded. Results should indicate the reliability of the technique in producing a similar activity pattern for the specific analyte at the different concentrations, but a unique pattern for each specific cell type to each specific analyte.

Smart Sensor System

The present invention provides a smart sensor system that comprises a microelectrode array with an enclosure coupled with a microfluidic system. The microelectrode array may be one known in the art such as a 5×5 microelectrode array, a 64 sensing site microelectrode array, a 96 sensing site microelectrode array, and the like. See Gross, G. W., et al. (1997) European Journal of Cell Biology 74:36-36; and Csicsvari, J., et al. (2003) J. Neurophysiology 90 (2):1314-1323, which are herein incorporated by reference. The enclosure is preferably made of a chemically and biologically inert material such as silicon, platinum, steel, titanium, cobalt-based alloys, titanium-based alloys, ceramics such as those comprising Al₂O₃, TiO₂, SiO₂, Fe₂O₃, and the like, carbon including graphite and glassy carbon, and the like.

A fluid sample to be tested can be supplied to the sensor system using methods known in the art. In preferred embodiments, the fluid sample is introduced to the sensor system through a suction system and a filter that will trap undesired agents such as atmospheric dust. The filtered fluid sample is then mixed with a buffer solution obtained from a microreservoir using a MEMS mixer. The MEMS mixer provides a rapid and close to homogeneous mixing of a sample and the buffer solution. In preferred embodiments, the dimensions and design of the MEMS mixer are modified to deliver the mixture to the cells and the sensor system at optimum rates. In preferred embodiments, the MEMS mixer is an interdigitated MEMS mixer having two inlets and one outlet with one inlet connected to the outlet of the filter and the other inlet connected to a microreservoir containing the cell growth medium to promote homogeneous mixing. Homogeneous mixing is essential for the quick uptake of the sample which will aid in rapid analysis of the analytes.

The buffer solution containing the atmospheric analytes is then supplied to the microelectrode array through a microfluidic inlet channel at a rate of about 30 μl/min. In preferred embodiments, the microfluidic inlet and outlet is able to support a flow rate of about 40 μl/min. The composition of the microfluidic channels may be polydimethylsiloxane (PDMS) which has a tendency to expand. Therefore, the dimensions and volume of the microfluidic channels may be optimized using methods known in the art in order to avoid back pressure and channel rupture.

The buffer solution in the enclosure may be removed by using a microfluidic outlet with a pump out rate of about 40 μl/min. The electrical activity from the patterned cells over the electrodes is measured from electrode pads. The data acquisition may be performed digitally using multi-channel digital oscilloscopes that are controlled through LabVIEW. Real time signal processing is performed and the acquired data is basically comprised of frequency domain transformations in order to create SPVs which are then processed by the developed performance model to pick up the eigen values unique to a specific analyte.

The data is then compared with a library of SPVs to determine the analyte or analytes present in the fluid sample. In case of the detection of an unknown analyte in a fluid sample the genre of analyte can be determined by comparing the SPV of the unknown analyte with similar SPVs in the library. FIG. 16 is the schematic representation of the prototype of the sensor system.

In preferred embodiments, the physiology of the isolated cells should remain constant over various experimental runs and conditions. Therefore, great care must be given to the production, storage and maintenance of physiologically representative mammalian cells for use in portable devices. The cells used in the sensors of the present invention may be cultured and maintained using methods known in the art. Additionally, the cells may be maintained in stasis using methods known in the art.

The fundamental requirement for the single cell based biosensor is a platform over which single cells can be immobilized and positioned. In preferred embodiments, the platform comprises conducting microelectrodes. The geometry of the design of the platform is such that the diameters of the electrodes are about 3 to about 4 times larger than the cell diameter and the distances between the electrodes are about 8 to about 10 times larger than the cell diameter. In preferred embodiments, the electrical signals measured from one electrode does not interact with the signals obtained from the nearest neighboring electrode.

In preferred embodiments, the electrodes are made of a material that is chemically and biologically inert so that the electrodes not react with the fluid samples and analytes that are to be tested. In preferred embodiments, the electrodes are highly conductive as the principle of cell isolation is primarily dependant upon the speed of variation of the strength of the applied electrical fields. Preferably, the material is a metal such as platinum, tin, titanium, and the like. In preferred embodiments, the sensor is packaged to provide a stable local microenvironment for portability and operability in field conditions.

In preferred embodiments, the sensor is a 5×5 multiple microelectrode array, with platinum electrodes (about 80 μm in diameter and about 200 μm center-to-center spacing) covering an area of about 0.88 mm . The electrodes are connected to platinum electrode pads (about 120 μm× about 120 μm) through platinum electrode leads (about 6 μm, thick). The sensor may be integrated with a chemically and biologically inert enclosure (about 0.16 mm× about 0.16 mm× about 0.25 mm) to provide or maintain a stable local microenvironment. A measuring or monitoring system known in the art may be employed measure or monitor electrical and/or optical changes which may be simultaneous or consecutive. Confocal optical imaging may be used to identify the receptor proteins involved in the modulation of the electrical activity. Computer programs and software known in the art may be used to analyze the electrical and optical changes and to determine SPVs.

Other Embodiments And Considerations

The reactant components are placed on a microelectrode array according to the present invention and then preferably immobilized. As used herein, “immobilize” or a grammatical equivalent thereof, means that the reactant component is fixed relative to the microelectrode. For example, a reactant component may be attached to a linker moiety, the reactant component may be embedded within a matrix of a linker moiety, or any combination thereof, and the linker moiety is attached to the microelectrode. Alternatively, the reactant component may be directly fixed to the electrode.

Methods for fixing a reactant component directly or indirectly to a microelectrode are known in the art and include methods that make use of covalent bonding, ionic bonding, van der Waals forces, hydro-phobic/philic interactions, biomolecule interactions (such as avidin-streptavidin interactions), polymerization and the like.

The microelectrode devices of the present invention may comprise more than one cassette. For example, a microelectrode device of the present invention may further include a “sample treatment” cassette that interfaces with a separate “detection” cassette; a raw sample is added to the sample treatment cassette and is manipulated to prepare the sample for detection, which is removed from the sample treatment cassette and added to the detection cassette. There may be an additional functional cassette into which the device fits; for example, a heating element or a data recording element. In some embodiments, the cassettes are removably attached to each other. See e.g. U.S. Pat. No. 5,603,351, and PCT/US00/33499, which are herein incorporated by reference.

The substrates of the invention can form microfluidic cassettes or devices that can be used to effect a number of manipulations on a sample to ultimately result in cell detection or quantification. These manipulations can include cell handling (cell concentration, cell lysis, cell removal, cell separation, and the like), separation of the desired cell from other sample components, chemical or enzymatic reactions on the cell, detection of the cells or other components, and the like. The microelectrode devices of the invention can include one or more wells for sample manipulation, waste or reagents; microchannels to and between these wells, including microchannels containing electrophoretic separation matrices; valves to control fluid movement; on-chip pumps such as electroosmotic, electrohydrodynamic, or electrokinetic pumps; and detection systems. The microelectrode devices of the present invention may be configured to manipulate one or multiple samples or analytes.

Suitable substrates include, silicon, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, Kovar™, Kevlar™, Kapton™, Mylar™, brass, sapphire, and the like. High melting borosilicate or fused silicas may be preferred for their UV transmission properties when any of the sample manipulation steps require light based technologies. In addition, portions of the internal surfaces of the microelectrode device of the present invention may be coated to confer desired properties such as reduce non-specific binding, allow the attachment of binding ligands, biocompatibility, flow resistance, and the like. In some embodiments, the substrate of the microelectrode device of the present invention preferably comprises silicon or glass. In some embodiments, the substrate comprises printed circuit board (PCP) materials including fiberglass, ceramics, glass, silicon, mica, plastic (including acrylics, polystyrene and copolymers of styrene and other materials, polypropylene, polyethylene, polybutylene, polycarbonate, polyurethanes, Teflon®, and derivatives thereof, and the like), combinations thereof, and the like.

The microelectrode devices of the present invention may be made according to methods known in the art. See WO96/39260, directed to the formation of fluid-tight electrical conduits; U.S. Pat. No. 5,747,169, directed to sealing; EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071,531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,795,453; 5,681,484; and 5,603,351, all of which are herein incorporated by reference. Suitable fabrication techniques again will depend on the choice of substrate and include various micromachining and microfabrication techniques, including film deposition processes such as spin coating, chemical vapor deposition, laser fabrication, photolithographic and other etching techniques using either wet chemical processes or plasma processes, embossing, injection molding and bonding techniques known in the art.

In addition, it should be understood that while most of the discussion herein is directed to the use of planar substrates with microchannels and wells, other geometries can be used as well. For example, two or more planar substrates can be stacked to produce a three dimensional device, that can contain microchannels flowing within one plane or between planes; similarly, wells may span two or more substrates to allow for larger sample volumes. Thus for example, both sides of a substrate can be etched to contain microchannels. See U.S. Pat. Nos. 5,603,351 and 5,681,484, which are herein incorporated by reference.

The microelectrode devices of the invention may include at least one microchannel or flow channel that allows the flow of sample from the sample inlet port to the other components or modules of the system. The collection of microchannels and wells is sometimes referred to in the art as a “mesoscale flow system”. As will be appreciated by those in the art, the flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example the sample inlet port and a reagent storage module, may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used.

In some embodiments, the microelectrode devices of the present invention are designed on a scale suitable to analyze nano- and microvolumes, although in some embodiments large samples (e.g. cc's of sample) may be reduced in the microelectrode device to a small volume for subsequent analysis. In addition to the flow channel system, the microelectrode devices of the invention include one or more components, herein referred to as “modules” or “cassettes” which include sample inlet ports; sample introduction or collection modules; cell handling modules, for example, for cell lysis, cell removal, cell concentration, cell separation or capture, cell growth, and the like; separation modules, for example, for electrophoresis, dielectrophoresis, gel filtration, ion exchange/affinity chromatography (capture and release) and the like; reaction modules for chemical or biological alteration of the sample, including amplification of the nucleic acids, chemical, physical or enzymatic cleavage or alteration of the sample components, or chemical modification of the target; fluid pumps; fluid valves; thermal modules for heating and cooling; storage modules for assay reagents; mixing chambers; detection modules, and the like.

In some embodiments of the present invention, the microelectrode device of the present invention comprises at least one reference or control electrode. In some embodiments, interaction and binding of reactant components are detected using AC impedance, impedance spectroscopy, cyclic voltammetry, AC voltammetry, pulse voltammetry, square wave voltammetry, AC voltammetry, hydrodynamic modulation voltammetry, conductance, potential step method, potentiometric measurements, amperometric measurements, current step method, other steady-state or transient measurement methods, and combinations thereof.

Performance Model

It is important to determine the sensing threshold and sensing capabilities of a cell for known analytes. Therefore, the present invention also provides performance models and methods of making performance models for SPV analysis. For example, a performance model based on neuronal behavior may be developed in order to determine the sensing threshold and sensing capabilities of a single neuron for known analytes.

This is the first step towards the ultimate goal of modeling the signature pattern vectors of cell types for unknown analytes in order to determine accurately the genre of the unknown analyte based on the generated signature pattern vector by comparing the experimentally obtained SPV with a modeled SPV. In preferred embodiments, the model describes the transmembrane electrical potentials in a cell in terms of the cell's ionic basis. The fundamental picture to be developed is that of a relaxation circuit activated primarily by modulations in membrane conductance. The transmembrane potentials are maintained by differential concentrations of ions and seared by ionic currents; molecular gating processes actively control the membrane conductance modulations during synaptic activation, the generation of action potentials and various other active membrane processes.

Resting Potential Model Based On the Goldman Equation

The primary model for the resting potential in a cell, such as a neuron, is expressed by the Goldman Equation as follows:

$${V}_{G} = \frac{kT}{q} In \frac{{{P}_{k} [ K ]}_{0} + {{P}_{Na} [ Na ]}_{0} + {{P}_{cl} [ Cl ]}_{i}}{{{P}_{k} [ K ]}_{i} + {{P}_{Na} [ Na ]}_{i} + {{P}_{cl} [ Cl ]}_{0}}$$

wherein the
P's are permeability of the membrane (proportional to conductance) to the different ionic species;
[ ] is the concentration of the different ionic species in the intracellular and extracellular fluid;
k is Boltzmann's constant,
T is the temperature; and
q is the unitary electrical charge. See Bard, A. J. and Faulkner, L. R., (1980) Electrochemical methods, John Wiley & Sons, Inc., New York, which is herein incorporated by reference.

The idea underlying the Goldman Equation is that when the transmembrane potential is fixed at its resting value, the total transmembrane current due to diffusion along concentration gradients and to constituent electric field gradients must sum to be zero.

The significance of this model is that it allows one to make precise, quantitative, causal predictions regarding modulations in trans-membrane electrical potentials in neurons as they are influenced by variations in the constituency of the intra- and extracellular fluids and by modulations in the permeability of the membrane to the various relevant ions.

Membrane Model

An electrical model for a lipid bilayer membrane that is permeable to an arbitrary number of ionic species, taking account of current components. The capacitive component comes from a current density as follows:

$${J}_{cap} = C \frac{ⅆ v}{ⅆ t}$$

$$C = \frac{k {ɛ}_{0}}{d}$$

wherein
C is the capacitance per unit area of the bilayer;
G_{Na} is the maximum sodium/potassium conductance per unit area; and
Gₖ is the maximum sodium/potassium permeability per unit area. See Bockris and Reddy (1970) Modern Electrochemistry, Plenum Press, New York, which is herein incorporated by reference.

In addition to the capacitive current, there is also an ionic current for each species of ion that is able to pass through the membrane as shown in FIG. 16.

In general, the total ionic current through a membrane is given by the following expression:

$$J = C \frac{ⅆ v}{ⅆ t} + {J}_{Na} + {J}_{K} + {J}_{Ca} + … + etc$$

Action Potential Model

The action potential model describes the transmembrane potential in the course of single-action potentials in terms of underlying membrane conductance modulations. The action potential model embodies and describes quantitatively the causal mechanism underlying the generation of action potentials. On the other hand, the model does not embody a satisfactory picture of the biophysical basis of the membrane conductance modulations in terms of more fundamental molecular events.

The total ionic current per unit area across the membrane is provided as follows:
Jᵢₒₙ=G_{Na}m³h(V−V_{Na})+G_{K}n⁴(V−V_{K})+G_{L}(V−V_{L})
wherein
G_{Na} is the maximum sodium/potassium conductance per unit area;
Gₖ is the maximum sodium/potassium permeability per unit area;
m or n is the “sodium/potassium turn on” variable; and
h is a “sodium turn off” variable;
m, h, and n are functions of both V and t, and the last term is a small leakage current, accounting for ionic current missed by the direct measurements of sodium and potassium components.
   Circuit Model For Synaptic Activation

The model for synaptic activation is the fundamental biophysical model of the ionic basis of neuro-electric activity. This model is based on the ionic gradients established due to the synaptic interaction among neurons by elucidating the electrophysiological mechanisms of direct chemically mediated synaptic activation, and integrating these within the ironically mediated relaxation circuit model to produce a dynamic model for information processing in neurons and, by implication, for neuronal networks. See for example FIG. 17.

Threshold Variation Model

The model for threshold voltage analysis uses a fixed threshold for comparison with a graded generator potential to determine firing. However, in real neurons excitability and thresholds do vary and a number of models of such variations have been presented. The triggering of action potentials is caused by the excursion of membrane conductance to sodium. Therefore, fundamental understanding of threshold variation will be dependent on understanding of process at the molecular level.

The following equation is for transmembrane potential E as determined by the conventional equivalent circuit model:

$$\frac{ⅆ E}{ⅆ t} = - \frac{E}{t} + I$$

The following second equation is for the threshold U considered as a continuous function of time:

$$\frac{ⅆ U}{ⅆ t} = \frac{( U - {U}_{0} ) + CE}{{T}_{U}}$$

The essential mechanism is that the threshold is driven away from its resting level according to the trans-membrane potential.

The variation in the transconductance determines the variation in the electrical activity of the neurons. Hence the response of a neuron to a specific analyte is triggered by the variation in the transconductance. This in turn generates the signature pattern vector. The method of variation of transconductance depends on the nature of the specific analyte. This can be modeled using the modeling components described in the previous sections.

The models of the present invention may be used to determine the cell types which are most sensitive to the certain analytes. The models of the present invention may be used to analyze the ion channels and ion gradients responsible for causing the electrical potentials across cell membranes. The models of the present invention may be used to determine the minimum concentrations of given analytes produces an observable change in the cell. The models of the present invention may be used to study the effects of given analytes on cellular growth.

The mechanism of how an analyte affects the ion flux of a given cell may be studied using the models of the present invention. For example, biocompatible voltage sensitive dyes that are sensitive to a specific ion flux (Jimbo, Y. et al. (1993) IEEE Trans. in BioMed. Engr. 40 (8):804-810, which is herein incorporated by reference) may be added to a cell culture and incubated so that the cells uptake the dye and express it only when a particular ion flux responsible for the change in the electrical activity is achieved. Then the receptor protein that underwent the modification in response to the analyte can be identified and studied. The receptor protein may be identified using methods known in the art, such as immunohistochemical staining of the cells.

Once the receptor protein is identified, the receptor may be isolated or cloned using methods known in the art. The method of extraction should be mild enough to preserve protein structure, yet tough enough to solubilize cellular membranes and other proteins not of interest.

Isolated or recombinant receptor proteins responsible for ion flux may be integrated on a protein microchip using microstamping techniques known in the art, thereby eliminating the need for using whole cells in CBBs. Thus, such PBBs will be portable and easy to use in field conditions.

### EXAMPLE 1

### Single Cell Positioning

Neurons were separated from glial cells of a neuronal cell culture and later positioned on the electrodes using an alternating current field. Specifically, single neurons are separated from a co-culture of glial cells and positioned over microelectrodes using dielectrophoretic forces. Dielectrophoresis is the motion of particles caused by the dielectric polarization effects in non-uniform electric fields. Alternating current (AC) fields of a wide range of frequencies were used to generate the inhomogeneous field. Due to their highly dielectric membrane properties, cells experience dielectrophoretic forces under the influence of a gradient electric field. The dielectrophoretic force acting on a cell of radius, r, suspended in a medium of dielectric permittivity εₘ is given by
F_{DEP}=2πr³εₘα∇E²
where α is a parameter defining the effective polarizability of the particle and the factor ∇E² is proportional to the gradient and the strength of the applied electric field. The polarizability parameter varies as a function of the frequency of the applied field strength and depending on the dielectric properties of the cell and the surrounding medium takes on the value between about +1.0 to about −0.5. A positive value of the polarization factor leads to an induced dipole moment over the cell membrane aligned along the direction of the field and produces a positive dielectrophoretic force. This occurs when the effective polarization of the cell is greater than the surrounding medium and causes the cells to move to the regions of high electric fields. Similarly, a negative value of the polarization factor causes the induced dipole moment align in the direction opposite to the applied field, which results in a negative dielectrophoretic force and causes the cells to move to regions of low electric fields.

Individual neurons were separated and patterned onto the microelectrodes due to the effect of a positive dielectrophoretic force on the neurons and a simultaneous negative dielectrophoretic force on the glial cells. The parameters for achieving this state are about 8 volts peak to peak voltage (Vₚₚ), frequency of about 4.6 MHz, and a conductivity of about 1.2 mS/cm. Single neuron positioning was achieved within about 3 to about 5 minutes of the application of the gradient electric field, for n=15, where n indicates the number of experimental trials. The technique of incorporating dielectrophoresis ensures the localization of individual neurons of the same biological state and viability over the array of test sites that yields comparable signals during sensing. This enables the minimization of false alarms due to potential variation among the cells under testing.

### EXAMPLE 2

### Membrane Excitability And Stain-Free Chemical Sensing

Extracellular signals from individual neurons due to the action of a specific chemical analyte may be analyzed further to understand the chemical type and the cellular response relationship. Here, single neuron based sensor's response and its sensitivity is determined by statistical reconstruction and enhancement of the acquired experimental data. Each chemical was characterized by a unique SPV obtained from the integrated processing of the modified extracellular action potential in the frequency domain (FFT) as well as the time domain (WT).

This technique has been used for highly sensitive detection of a broad spectrum of chemicals ranging from behavior altering agents like ethanol, whose action is analogous to the effect of pentobarbitone and ketamine; environmentally hazardous agents like hydrogen peroxide, which affects the cell membrane in a manner that mimics carcinogenic chemicals like rotenone and ethylene diamine tetra acetic acid (EDTA), which encompasses a class of non biodegradable phosphate alternative compounds to physiologically harmful agents, pyrethroids which causes effects similar to those obtained due to the action of dichlorodiphenyltrichloroethane (DDT) and other commercial pesticides. Most notably, the effectiveness of the single neuron sensor in regaining its initial physiological state has been demonstrated and multiple agents have been identified consecutively, referred to as “cascaded sensing”. This ability to monitor real time environmental changes which is invaluable for field testing purposes is exhibited through this sensing technique.

Integrated signal processing yielded an SPV specific to every chemical analyte. The SPV provides unique functional data corresponding to the physiological state of a single neuron due to the action of a particular analyte at a specific concentration. Each SPV comprises of certain frequency states, which are analyte specific and having maximum relative amplitudes that are generated concurrently in the sensing cycle; these correspond to the neuron's modified stable burst rate. Extracellular signal amplitudes greater than about 50 μV are considered to be accurate measures of the cell response due to a specific chemical analyte as the magnitude of signals due to non-specific interactions and noise signals are smaller than this value. See Jimbo Y., etal. (1993) IEEE Trans. Biomed. Eng. 40:804-810, which is herein incorporated by reference. Hence, processing was performed on acquired signals greater than this threshold value during the data analysis.

In the first stage, control experiments were performed in which a neuron was exposed to the sensing buffer in the absence of chemical analytes, and the extracellular signal was recorded and analyzed to generate the initial (background) SPV pertaining to neuron's characteristic burst rate depending on its physiological condition. FFT analysis extracted the characteristic burst frequency from the firing pattern. The characteristic burst frequency was determined to be at 626 Hz that corresponds with neuronal electrical activity determined from other topographical methods known in the art. See Kamioka H., et al. (1996) Neurosci. Lett. 206:109-112; and Maher, M. P., et al. (1999) J. Neurosci. Meth. 87:45-56, which are herein incorporated by reference.

In the second stage, chemical analytes were first premixed individually with the sensing buffer and introduced into the sensor system. The modified electrical activity due to presence of chemical analytes was recorded. Testing of a specific chemical analyte was performed in a cyclic manner with each cycle comprising of three phases. The time duration of each phase was on an average of about 60 seconds. The data presented herein is averaged over fifteen cycles (n=15). The action of each chemical analyte at decrementing concentration ranges (step size in the higher concentration range: 500 ppm, lower concentration range (<1000 ppm:50 ppm) was determined by monitoring the electrical activity at 5 seconds intervals for the first 30 seconds and then at 30 seconds intervals over a period of 180 seconds. This constitutes a single sensing cycle.

In the presence of each specific chemical analyte (ethanol concentrations ranging from about 5000 ppm to about 5 ppm, hydrogen peroxide: about 5000 ppm to about 10 ppm, pyrethroids: about 5000 ppm to about 250 ppb, EDTA: about 5000 ppm to about 150 ppm), pronounced modifications in the extracellular action potentials were observed. The detection limits for a single neuron was as follows: ethanol about 9 ppm, hydrogen peroxide about 19 ppm, pyrethroids about 280 ppb, and EDTA about 180 ppm. The lowest single neuron sensitivity as estimated theoretically by the existing methods of averaging and iteration indicate the lowest concentrations determined that are ethanol (MW=46.07) about 25 μM (Maldve et al. (2002) Nat. Neurosci. 5:616-641 , which is herein incorporated by reference) as compared to the experimentally obtained detection limit of about 2.17×10⁻¹²M, hydrogen peroxide (MW=34.01) about 15 nM as compared to about 2.94×10⁻¹²M (Bruijin etal. (1998) Science 281:1851-1854, which is herein incorporated by reference), pyrethroids (MW=38.3) about 12 pM as compared to about 3.05×10⁻¹⁴ M (Wegeroff (1997) 1^{st} ed. Thieme, Stuttgart, which is herein incorporated by reference), EDTA (MW=292.2) about 15 pM to about 3.42×10⁻¹³M (Subramaniam, J. R., etal. (2002) Nature Neurosci. 5:301-307, which is herein incorporated by reference).

The SPV is unique to a specific chemical analyte and remains unchanged for varying concentrations of the specific analyte. The SPV obtained from a single neuron in the absence of a chemical analyte indicates the initial control characteristic burst rate of 626 Hz. Addition of ethanol leads to its binding to M1 and M2 regions on the outside face of the GABA_{A} and glycine receptor gated Cl⁻ ion channels. See Maldve R. E., et al. (2002) Nat. Neurosci. 5:641-616, which is herein incorporated by reference. This increases the duration of the channel openings causing a strong inhibitory ionic current associated with Cl⁻ influx and decreased the frequency of firing to 314 Hz. Addition of hydrogen peroxide causes its binding to the α subunit of the APMA gated Na⁺ ion channels which produces a rapid ionic depolarization current. It simultaneously acts upon the NMDA gated channels which triggers the entry of Ca⁺⁺ ions into the cell, which causes the transmembrane release of glutamate and a steep increase of intracellular levels of Ca⁺⁺. See Bear M. F., et al. (1999) NEUROSCIENCE: EXPLORING THE BRAIN Lippincott, Williams and Wilkins, Baltimore, Md., 2nd ed. pp. 147, which is herein incorporated by reference. The low frequency eigen vectors (175 Hz, 227 Hz, and 349 Hz) indicate the initial activation of APMA gated channels due to initial short binding transients of hydrogen peroxide. The mid frequency eigen vector (453 Hz) corresponds to the activation of NMDA gated channels and the longer duration of binding of hydrogen peroxide to NMDA receptors. The high frequency eigen vectors (749 Hz and 975 Hz) correspond to the induced excitotoxicity. See Stout A. K., et al. (1998) Nature Neurosci. 1 (5):366-373, which is herein incorporated by reference.

Addition of pyrethroids results in the activation of the NMDA gated channels. The negative charge along the membrane surface induces the binding of Mg⁺⁺ ions causing the clogging of the channels thus preventing the flow of Na⁺ and K⁺ ions. See Bear M. F., et al. (1999) NEUROSCIENCE: EXPLORING THE BRAIN Lippincott, Williams and Wilkins, Baltimore, Md., 2nd ed. pp. 147, which is herein incorporated by reference. These results in the reduction of the depolarizing ionic current reducing the firing rate to 514 Hz. Addition of EDTA causes its binding to the GABA_{A} as well as the NMDA receptor gated Cl⁻ and Na⁺ ion channels, respectively. See Subramaniam J. R., etal. (2002) Nature Neurosci. 5:301-307, which is herein incorporated by reference. The GABA_{A} activation produces a hyperpolarizing current resulting in low frequency bursting at 227 Hz and NMDA activation produces a high frequency burst of 873 Hz.

The generated SPV for each chemical analyte produced the same eigen vectors for varying concentrations of the specific chemical analyte. This ensured that the method of the present invention is concentration independent and is highly specific to the particular chemical analyte. A variation in eigen value over the concentration range (5000 ppm to detection limit) was observed. The variation in concentration was inversely proportional to the amplitude of the generated signal. The percentage reduction of signal amplitudes from the detection limit to the peak concentration for various chemicals was determined to be as follows: ethanol about 52% with about 8.2%±0.12% per 1000 ppm, hydrogen peroxide about 38% with about 7.8%±0.14% per 1000 ppm, pyrethroids about 89% with about 12.2%±0.14% from 5000 ppm to 1000 ppm and about 3.1%±0.07% below 1000 ppm, EDTA about 67% with about 10.4%±0.11% per 1000 ppm, for n=15.

The response time of a single neuron to each specific chemical analyte was extracted via WT analysis. A “response time” as computed by WT is defined as the duration for the amplitude of the signal to change from the initial value to the extreme, for each chemical analyte. Simultaneous use of the band filtration technique ensured the elimination of low frequency noise (about 60 Hz or less). The response time of each chemical analyte as a function of its concentration was determined. The analysis indicated the response time of a single neuron to a specific chemical analyte to be inversely proportional to the concentration of the chemical analyte. The action of ethanol at its detection limits on the neuron cell membrane produces signal transience that is due to the modulation of Cl⁻ flow by the increased duration of channel openings. See Franks N. P. and Lieb W. R. (1997) Nature 389:334-335, which is herein incorporated by reference.

The response time was determined to be about 0.21 second. The response time due to hydrogen peroxide was determined to be about 0.07 second. The response due to hydrogen peroxide was the quickest as it acts upon the APMA gated channels which determine the excitatory response due to a rapid influx of Na⁺ ions. See Lee, M. S., et al. (2000) Nature 405:360-364, which is herein incorporated by reference. The response time for pyrethroids is determined to be the longest at the detection limit. It was calculated to be about 0.43 second. The large response time is associated with the slow activation of the NMDA gated ion channels and the resulting Mg⁺⁺ block responsible for low hyperpolarization. The response time for EDTA was determined to be about 0.27 second at the detection limit. The smooth transients observed in the waveform are due to the frequent switching of the Cl⁻ channels from open to closed states. See Sun, Y., et al. (2002) Nature 417:245-253, which is herein incorporated by reference. The percentage increase in response time from peak concentration to the sensitivity limits was as follows: ethanol about 1.6%, hydrogen peroxide about 1.8%, pyrethroids about 1.7%, EDTA about 1.8%, for n=15. The response time for each chemical was determined over the concentration ranges from about 5000 ppm/mm² to the detection limit for each chemical, for n=15. There was an inverse relationship between the concentration of a chemical analyte and the associated response time. The average variation of the absolute amplitude of the signal was about ±1.73% of the average value.

To rule out the possibility of non-specific interactions, the electrical activity due to the effect of the sensing medium on the electrode was recorded. Spectral analysis indicated a low frequency signal (less than about 60 Hz) which was filtered out in the WT analysis. Hence, this does not induce any modifications to the responsiveness or the detection limit. This is a fail safe technique as the modification of the electrical activity of an individual neuron and the generation of the associated SPV occurs only in the presence of a chemical analyte with a concentration above the detection limit which then would affect the physiological behavior of the cell as determined by the response time. Also the presence of individual cells of similar physiological conditions ensures the reproducibility of the sensing pattern.

### EXAMPLE 3

### Cascaded Sensing of Multiple Chemical Analytes

The sensing technique disclosed above was used to investigate the sensing of multiple chemical analytes interacting with a single neuron in a temporal manner also termed as “cascaded sensing”. This is used to establish a single neuron's function as a reusable sensor with the ability to distinguish between various chemical analytes, i.e. exhibit selectivity. The detection limits for individual chemicals act as the basis for determining the concentration of the specific chemical analytes used in cascaded sensing. Addition of the first chemical analyte approaching its sensitivity limit results in the acquisition of modified extracellular potential pertaining to the specific chemical analyte. The use of the chemical analyte close to its detection limit leads to the dissipation as well as metabolization of the chemical analyte within a single sensing cycle (180 seconds) that result in the reduction of the chemical analyte concentration below the detection threshold. See Kash T. L., et al. (2003) Nature 421:272-275, which is herein incorporated by reference. This is determined from the signature pattern that exhibits the restoration of the neuron's characteristic control firing frequency at the end of one cycle. Administration of the second chemical analyte results in a specific modification based on the latter. The frequency spectrum indicates the combination of eigen vectors unique to the second chemical analyte during the second sensing cycle generated after the addition of the new chemical analyte in cascade.

The chemicals evaluated were ethanol and hydrogen peroxide. The concentrations of the chemicals used were at their detection limits. The frequency spectrum analysis produced a signature pattern in the first 300 seconds that corresponded to the signature pattern associated due to the action of ethanol. After this time duration, it was estimated that the binding of ethanol to the α subunit of GABA_{A} receptor was weakened which induced the expression of the control characteristic frequency. The addition of hydrogen peroxide at this instant led to the generation of the frequency spectrum of hydrogen peroxide. All the eigen vectors corresponded to those obtained due to the isolated action of hydrogen peroxide except for the high frequency eigen vector of 749 Hz which underwent a 26 Hz shift up to 775 Hz, which is likely a result of the interaction of ethanol with hydrogen peroxide.

### EXAMPLE 4

### Calcium Imaging In Neuronal Cells

Ca²⁺ Indicator Loading

The freshly dissociated neurons were suspended in Krebs solution comprising: 118.8 mM NaCl, 25 mM NaHCO₃, 1.13 mM NaH₂PO₄, 4.7 KCl, 1.8 CaCl₂, 1.2 MgCl₂, 11.1 mM glucose and constantly gassed with about 95-5% CO₂ to pH 7.4. The neuron culture was then loaded with a saturated solution of Oregon Green 488 BAPTA-1, dextran linked with a molecular mass of 10 kDa (Molecular Probes Inc., Ore.), in 2.5% Triton X-100 at 37° C. The culture with the indicator was then incubated for 3 hours in the dark at room temperature to allow time for the indicator to travel along axons, and then washed for an additional 2 hours to remove any extracellular dye.

Stimuli And Recording

The neuron culture was mounted in a 2 ml organ bath and placed on the stage of a Leica TCS NT laser-scanning confocal microscope. The preparation was stimulated with the specific chemical analyte under study. The initial stimulus concentration was 5000 ppm and was then decremented in a stepwise manner to the lowest concentration for which the neurons showed a response for that specific chemical analyte that was previously determined in the electrical characterization experiments. Concentration of the chemical analyte added to the neuron cell culture was such that the stimulus was super threshold for the axon thus preventing variation in the Ca²⁺ transient due to the recruitment of unstimulated axons in the culture. The 488 nm wavelength of an Argon ion laser was used for exciting fluorescence. A 515 nm long pass emission filter was used. While detecting Ca²⁺ transients, sets of images were captured for 56 seconds every 3 minutes. This protocol prevented excessive photo bleaching and photo toxicity. All experiments were carried out at 33° C. in the presence of nifedipine (10 μM; an L-type Ca²⁺ channel blocker), prazosin (1 μM; a competitive α₁-adrenoceptor antagonist) and α,β-methylene ATP (1 μM; a P_{2X}-receptor desensitizing agent) to reduce or abolish contractions elicited by high frequency stimulation required for better imaging.

Immunohistochemical Staining of Neurons

The freshly dissociated neurons were plated in a 35-mm petri dish comprising neuronal basal medium supplemented with fetal bovine serum (FBS) with a cell density of 20% confluence. The neuronal basal medium was aspirated and the neurons were washed 1× with phosphate buffered saline (PBS), NaCl (0.15 M), Na₂HPO₄ (12 mM), KH₂PO₄ (2 mM), pH 7.4 and 1× with tris phosphate-buffered saline (TBS), NaCl (0.15 M). A paraformaldehyde solution (PFA; 4% w/v, Sigma, St. Louis, Mo.), buffer (0.1 M) pH 7.4, were added to the cells and incubated at room temperature for 10 minutes. Sheep anti rabbit IgG (AFF, pur): FITC (Serotec, Oxford, UK); was added to the neurons. The neurons were mounted to the Petri-dish utilizing PBS/glycerol-mounting medium, pH 7.4. Imaging was completed using a fluorescence microscope with FITC and rhodamine filter blocks.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A combination consisting essentially of (a) a single reactant component which is a microorganism or a cell immobilized directly to (b) a single electrode.

2-4. (canceled)

5. The combination of claim 1, wherein the microorganism is a bacterium.

6. The combination of claim 5, wherein the bacterium is E. coli.

7. The combination of claim 1, wherein the cell is an osteoblast, a glial cell, or a neuron.

8. The combination of claim 1, wherein the single electrode is made of iridium, platinum, palladium, gold, silver, copper, mercury, nickel, zinc, titanium, tungsten, aluminum, carbon, graphite, a metal oxide, a conducting polymer, a metal doped polymer, a conducting ceramic, a conducting clay, or a combination thereof.

9. The combination of claim 1, wherein the single electrode has a diameter of about 60 μm to about 80 μm.

10. The combination of claim 1, wherein the single electrode has a diameter of about 40 μm to about 60 μm.

11. The combination of claim 1, wherein the single electrode has a diameter of about 20 μm to about 40 μm.

12. The combination of claim 1, wherein the single electrode is placed on or immobilized on a substrate.

13. The combination of claim 12, wherein the substrate is made of silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

14. A plurality of the combination of claim 1.

15. A device comprising at least one combination according to claim 1.

16. (canceled)

17. The device of claim 15, wherein the combinations may be the same as or different.

18. (canceled)

19. The device of claim 15, and further comprising a substrate upon which the combination is placed or immobilized.

20. The device of claim 19, wherein the substrate is made of silicon, silicon dioxide, silicon nitride, glass, fused silica, borosilicate, gallium arsenide, indium phosphide, aluminum, ceramics, polyimide, quartz, a plastic, a resin, a polymer, a superalloy, zircaloy, steel, gold, silver, copper, tungsten, molybdeumn, tantalum, Kovar™, Kevlar™, Kapton™, Mylar™, Teflon®, brass, sapphire, fiberglass, a ceramic, mica, or a combination thereof.

21. The device of claim 15, and further comprising a permeation layer, an electrode pad, a measurement system, an environment chamber, a pulse generator, a micromanipulator, a CCD camera, a multichannel oscilloscope, a digital signal processor, a MEMS mixer, a suction system, a filter, a microreservoir, a microfluidic channel, a treatment cassette, a detection cassette, a data recording element, a reagent storage module, a mixing chamber, a reaction chamber, or combinations thereof.

22. A method of making the combination of claim 1, which comprises using an alternating current field to position the single reactant component over the single electrode.

23-24. (canceled)

25. A biosensor which comprises at least one combination according to claim 1.

26. A method of assaying, analyzing, or monitoring a target analyte which comprises contacting a sample suspected of having the target analyte with the combination of claim 1 and detecting a change or a result, if any.

27-28. (canceled)

29. A method of identifying an unknown analyte as a known analyte or being similar to a known analyte which comprises contacting a sample suspected of having the unknown analyte with the combination of claim 1, determining a signature pattern vector for the unknown analyte and comparing the signature pattern vector with the signature pattern vector of the known analyte or the signature pattern vectors in a signature pattern vector database.

30. A method of making a signature pattern vector database which comprises determining a plurality of signature pattern vectors for the plurality of claim 14.

31. A combination consisting of (a) a single reactant component which is a microorganism or a cell immobilized directly to (b) a single electrode.
